SEQUENCE LISTING (4 sequences)

SEQ ID NO: 1 - DNA, 19 bp, Artificial
  Forward primer for KRTCAP3
aggactgctggatcctctg

SEQ ID NO: 2 - DNA, 19 bp, Artificial
  Reverse primer for KRTCAP3
gcacctgctgtcctaaacc

SEQ ID NO: 3 - DNA, 30 bp, Artificial
  Forward primer for beta-actin
tgacggggtcacccacactgtgcccatcta

SEQ ID NO: 4 - DNA, 29 bp, Artificial
  Reverse primer for beta-actin
ctagaagcattgcggtggacgatggaggg

(19) United States
(12) Patent Application Publication
(10) Pub. No.: US 2011/0002938 A1
(43) Pub. Date: Jan. 6, 2011

(54) HEMATOPOIETIC CELLS EXPRESSING THE PROTEIN KRTCAP3 AND LIGANDS FOR THE PROTEIN KRTCAP3

(75) Inventors: Sergio Abrignani, Serre Di Rapolano (IT); Mariacristina Crosti, Milano (IT); Monica Moro, Pioltello (IT)

Correspondence Address: LUCAS & MERCANTI, LLP, 475 PARK AVENUE SOUTH, 15TH FLOOR, NEW YORK, NY, 10016, US

(73) Assignee: ISTITUTO NAZIONALE DI GENETICA MOLECOLARE- INGM, Milano (IT)

(21) Appl. No.: 12/920,164

(22) PCT Filed: Mar. 26, 2009
(86) PCT No.: PCT/IB09/05081

§ 371 Date: Sep. 14, 2010

(30) Foreign Application Priority Data
Mar. 27, 2008 (IT) MI2008A000508

(51) Int. Cl. A61K 39/395; C12N 5/078; C12N 5/0781; C12N 5/0783; G01N 33/567; A61K 35/12; A61K 38/17; A61P 37/06; A61P 29/00; A61P 7/06; A61P 37/04

(52) U.S. Cl. 424/153.1; 435/325; 435/7.24; 424/93.71; 424/184.1; 435/375

(57) ABSTRACT

The present invention relates to ex vivo hematopoietic cells characterized by the expression of the protein KRTCAP3 on the surface of said cells, to methods for isolating said cells and to ligands for KRTCAP3.

The present invention relates to ex vivo hematopoietic cells characterized by the expression of the protein KRTCAP3 on the surface of said cells, to methods for isolating said cells and to ligands for KRTCAP3. The gene KRTCAP3 is known in the art for its gene sequence. The gene for Homo sapiens is represented by GeneID 200634 according to Entrez gene designation (http://www.ncbi.nlm.nih.gov/entrez). The gene KRTCAP3 is present on chromosome 2. The acronym of the protein krtcap3/KRTCAP3 means keratinocyte associated protein 3.

The protein KRTCAP3 has a length of 240 aminoacids and is a “multi-pass membrane” protein.

The expression of KRTCAP3 was shown in skin, pancreas and keratinocytes with tests using techniques for measuring gene expressions.

There is a strong need in the art to improve the procedures for isolating and identifying specific cells belonging to the hematopoietic system.

There is also a strong need to improve the use of cells belonging to the hematopoietic system in the field of therapy/diagnosis/prognosis.

Eventually, there is a strong need in the art to be able to define the metabolic and/or physiological state of a cell belonging to the hematopoietic system. Assays for measuring the metabolic and/or mitogenic state of some cells belonging to the hematopoietic system are known in the art, in particular the interactions between cells of the immune system. For instance, the assay disclosed in Galli G., Nuti S., Tavarini S., Galli-Stampino L., De Lalla C., Casorati G., Dellabona P., Abrignani S., CD1d-restricted help to B cells by human invariant natural killer T lymphocytes. J Exp Med. 2003 Apr. 21; 197(8): 1051-7. Epub 2003 Apr. 14 enables to measure the interactions between T and B lymphocytes and the resulting metabolic and/or mitogenic states of B lymphocytes.

The Applicant has surprisingly found that the expression of KRTCAP3 on hematopoietic cells meets the above needs since the expression of KRTCAP3 and the resulting presence thereof on the surface of said cells are related to the mitogenic and/or metabolic state of hematopoietic cells.

The present invention is disclosed in the following detailed description as well as in the accompanying figures.

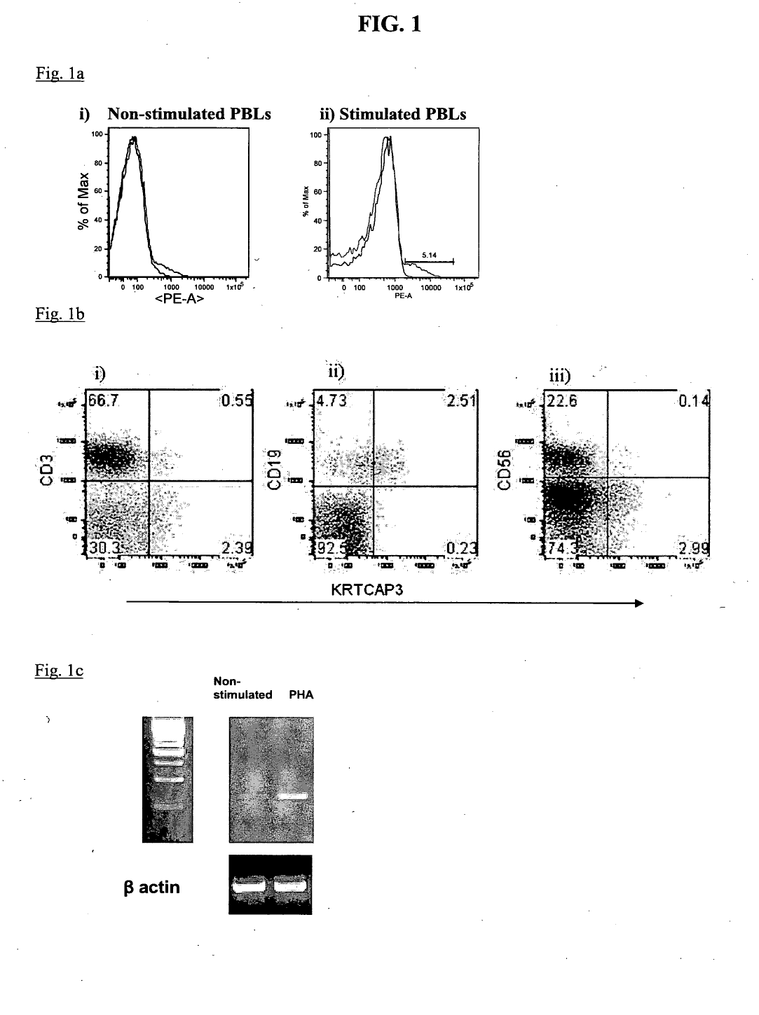

FIG. 1 shows the result of a test in which the distribution of KRTCAP3 on the surface of lymphocytes present in peripheral blood is detected by “Fluorescent-activated cell sorting” (FACS) (see Example 1 for the description of this method).

FIG. 1a shows the distribution of KRTCAP3 on the surface of peripheral blood lymphocytes (PBLs) detected using FACS, before and after stimulation with phytohemagglutinin (PHA) (FIG. 1a)i) and 1a)ii), respectively). PBLs are identified inside peripheral blood mononucleated cells (PBMCs) on the basis of physical parameters concerning size (Forward Scatter, FSC) and granulosity (Side Scatter, SSC). The graphs show two tracks, one of them for the control of the effectiveness of the antibody against KRTCAP3.

It can be seen that before stimulation PBL cells do not substantially express KRTCAP3 (the presence of the small peak is highly variable and has no statistical significance), and after stimulation with PHA 5.14% of PBL cells express KRTCAP3.

FIG. 1b shows the distribution of KRTCAP3 on the surface of specific lymphocyte sub-populations after stimulation with PHA. Said sub-populations are selected by means of markers present on the surface of said cells: CD3 for T lymphocytes, CD19 for B lymphocytes and CD56 for NK cells, represented in FIGS. 1b)i), b)ii) and b)iii), respectively.

In FACS diagrams the top quadrants show the specific sub-populations identified using said markers present on the surface of the cells selected from PBMCs, and the top right quadrant shows the percentage of said identified cells expressing KRTCAP3.

It can be seen that the relative numbers of cells having KRTCAP3 on the surface of T lymphocytes and NK cells are much smaller than the number of said cells not expressing KRTCAP3, so that these can be related to the background noise due to the method for producing antibodies for KRTCAP3 used in FACS test and have therefore no statistical significance. Conversely, it should be pointed out that the number of B lymphocytes expressing KRTCAP3 is quite large, 35% B lymphocytes present.

FIG. 1c shows the results of a RT-PCR test (see Example 1C) in which there is an increase of KRTCAP3 expression in PBMCs after stimulation with PHA. The expression of beta-actin is shown as test control. The left-hand column represents a standardized scale for measuring the size of the sequence (see Example 1).

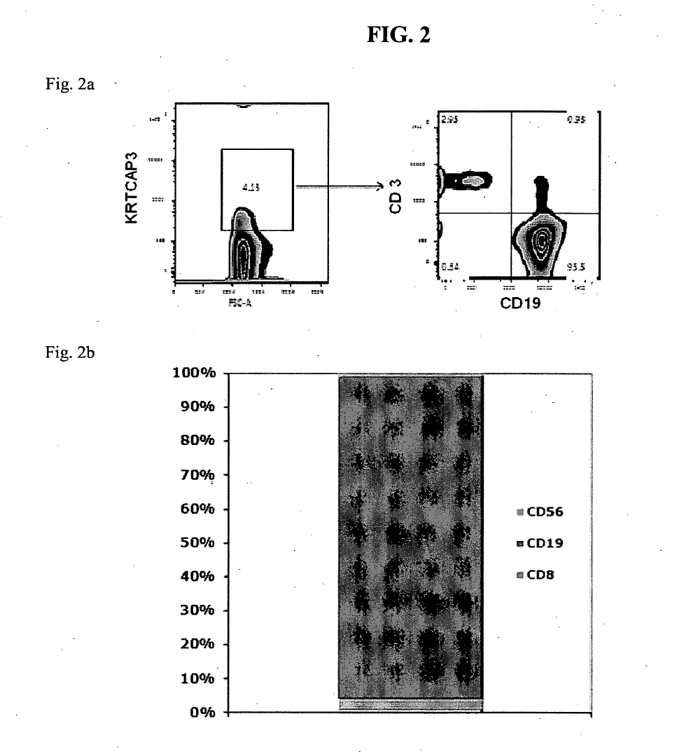

FIG. 2 shows the results of a test in-which the average distribution of KRTCAP3 on the surface of B lymphocytes present in the peripheral blood of 5 different donors is detected by “Fluorescent-activated cell sorting” (FACS) after stimulating B lymphocytes with phytohemagglutinin and interleukin 2 (PHA+IL-2) (see Example 1D for the description of this method). The results point out that 4.13% of the population of lymphocytes present in blood express KRTCAP3 after stimulation with PHA and IL-2 and 95.5% thereof are B lymphocytes. The remaining percentage can be related to background noise due to FACS test.

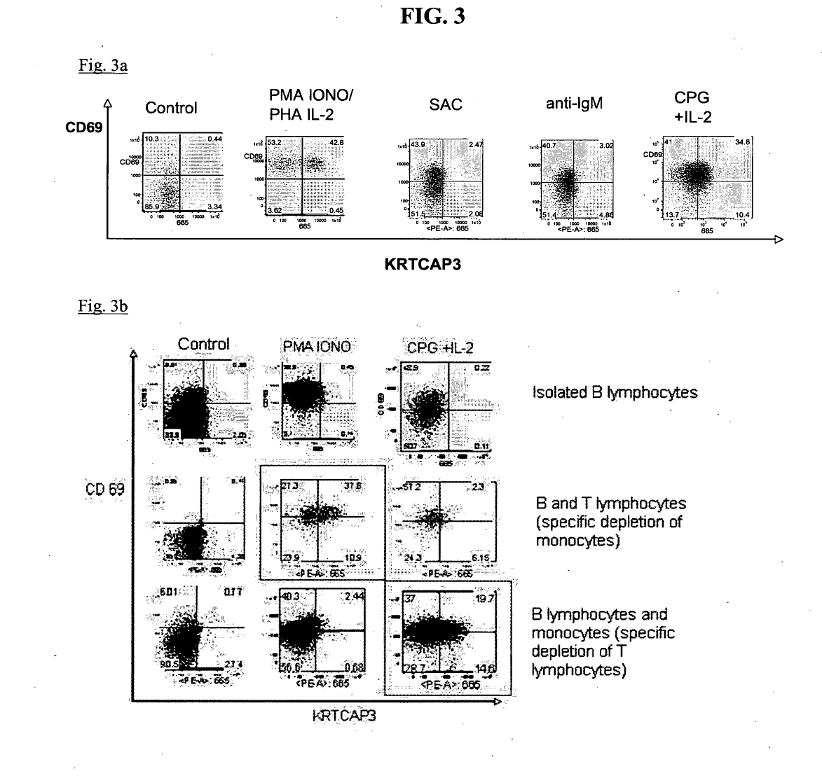

FIG. 3 shows the results of a set of tests in which the distribution of KRTCAP3 on the surface of B lymphocytes present in peripheral blood is detected by “Fluorescent-activated cell sorting” (FACS) (see Example 2 for the description of said method) 48 hours after stimulation with various metabolic and/or mitogenic cell stimuli or without stimulation (as control).

FIG. 3a shows the results of a set of tests evaluating the expression of KRTCAP3 on B lymphocytes 48 hours after stimulation of a group of PBMC cells with various metabolic and/or mitogenic stimuli.

Metabolic and/or mitogenic cell stimuli are phytohemagglutinin with interleukin 2 (PHA+IL-2), cytosin-phosphate-guanosin with interleukin 2 (CPG+IL-2), phorbol myristate acetate with ionomycin (PMA+IONO), cell lysate of Staphylococcus aureus (SAC) and M-type anti-Immunoglobulin antibodies (anti-IgM). B lymphocytes are identified by the expression of marker CD19 and FIG. 3 shows the analysis executed only on cells expressing such marker. Stimulated B lymphocytes are identified thanks to their expression of cell marker CD69. Therefore, the two top quadrants of each FACS diagram show together the total population of stimulated B lymphocytes. The top right quadrant of each FACS quadrant shows stimulated B lymphocytes also expressing KRTCAP3.

It should be pointed out that anti-IgM and SAC stimuli do not induce a significant increase of KRTCAP3 expression, whereas there is a high, statistically significant increase of KRTCAP3 expression on stimulated B lymphocytes when T lymphocytes are stimulated (with PHA+IL-2) or monocytes are stimulated (with CPG+IL-2). FIG. 3b shows the results of a set of tests evaluating the effect of various metabolic and mitogenic stimuli on the expression of KRTCAP3 on B lymphocytes isolated from the other PBMC populations by immunomagnetic separation (B cell isolation kit, Miltenyi), on B lymphocytes grown in presence of T lymphocytes only and on B lymphocytes grown in presence of monocytes only (see Example 2).

Metabolic and/or mitogenic cell stimuli used are cytosin-phosphate-guanosin with interleukin 2 (CPG+IL-2) and phorbol myristate acetate with ionomycin (PMA+IONO).

It should be pointed out that isolated B lymphocytes do not significantly increase the expression of the protein KRTCAP3 on their surface in response to these stimuli. It should also be pointed out that B lymphocytes grown in presence of T lymphocytes only increase the expression of KRTCAP3 in response to PMA+IONO but not to CPG+IL2, whereas B lymphocytes grown in presence of monocytes only increase the expression of KRTCAP3 in response to CPG+IL2 but not in response to PMA+IONO.

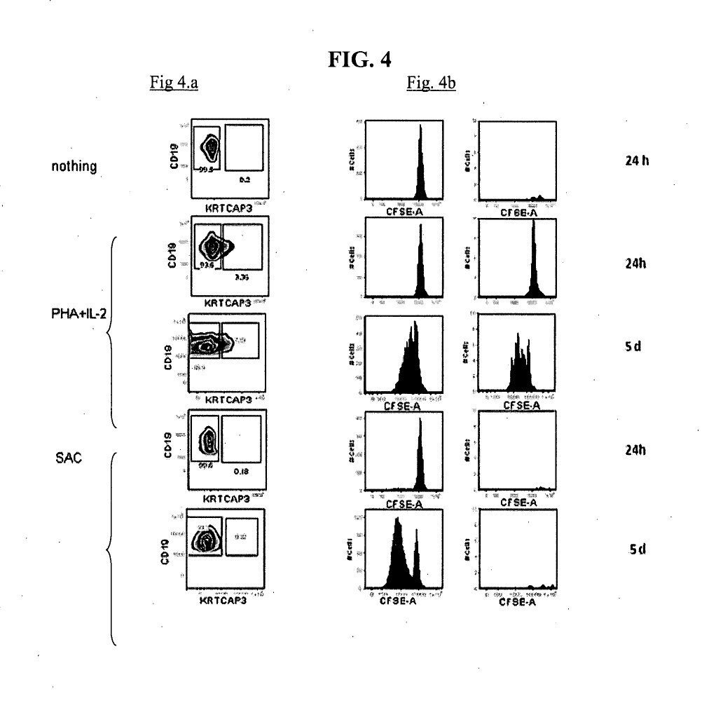

FIG. 4 shows the results of a test demonstrating the relation between the expression of KRTCAP3 in B lymphocytes and the mitogenic activity of said B lymphocytes, wherein mitogenic activity is evaluated in an independent manner as shown in Example 3.

The column in FIG. 4a shows FACS results determining the percentage of B lymphocytes (selected for expression of CD19) further expressing the protein KRTCAP3. It can be observed that without stimuli (negative control) or after 1 day or after 5 days of stimulation with SAC stimulator (positive control), no expression of KRTCAP3 is obtained. Conversely, after 1 day, and in particular after 5 days of stimulation with PHA+IL-2, a large population of B lymphocytes express KRTCAP3.

FIG. 4b shows the mitogenic activity evaluated in an independent manner using known coloring agent CFSE-A (5,6-carboxyfluorescein diacetate succinimidyl ester). The column on the left shows the peak of CFSE-A in B lymphocytes not expressing KRTCAP3 (represented by the left-hand quadrants of the corresponding FACS test). It can be observed that samples that are not stimulated or that are stimulated after 24 hours show a homogeneous peak of the coloring agent typical of undivided populations. Samples stimulated with PHA+IL2 or SAC after 5 days show a CFSE-A profile characterized by the presence of several peaks with lower fluorescence intensity, which indicates a significant mitogenic activity in said populations. From said expression of fluorescence in the samples and with a specific software (such as e.g. FlowJo (Treestar)) it can be inferred that B lymphocytes expressing KRTCAP3 have a higher mitogenic activity after 5 days of stimulation with PHA+IL-2 than those not expressing KRTCAP3.

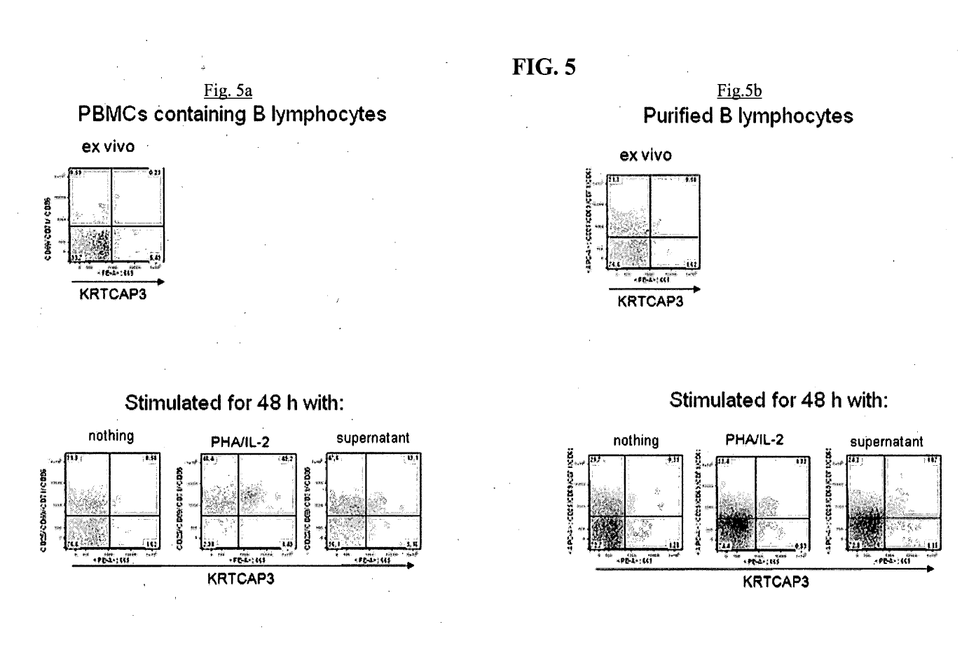

FIG. 5 shows the results of a test demonstrating that the activation of B lymphocytes by PHA+IL-2 (reference is made to the tests of Example 2C), which is then evaluated by means of KRTCAP3 expression, requires the physical presence of T lymphocytes and not only their presence in solution. The method applied is explained in Example 2C. The horizontal axis of the diagrams indicates KRTCAP3 expression, whereas the vertical axis indicates the expression of a series of lymphocyte activation markers (CD25, CD69, CD71, CD86) analyzed together.

FIG. 5a shows a series of tests made on B lymphocytes with other PBMC cells present, including T lymphocytes. Without stimuli after 48 hours there is no expression of KRTCAP3, but after stimulation with PHA+IL-2 there is a large presence of KRTCAP-3. The FACS result designed supernatant points out that B lymphocytes are not stimulated only by the supernatant (a definition of supernatant is given in Example 2C). Therefore, FIG. 5a shows that the physical presence of other PBMC cells is required in order to activate B lymphocytes with PHA+IL-2, and that these cannot be activated only by way of soluble factors.

FIG. 5b shows the negative control, in which B lymphocytes are stimulated without PBMC and thus without T lymphocytes. As was already shown in FIG. 4, the activation of B lymphocytes isolated with PHA+IL-2 is not sufficient to induce KRTCAP3 expression, all the more so the activation by way of supernatant or not has no effect on KRTCAP3 expression.

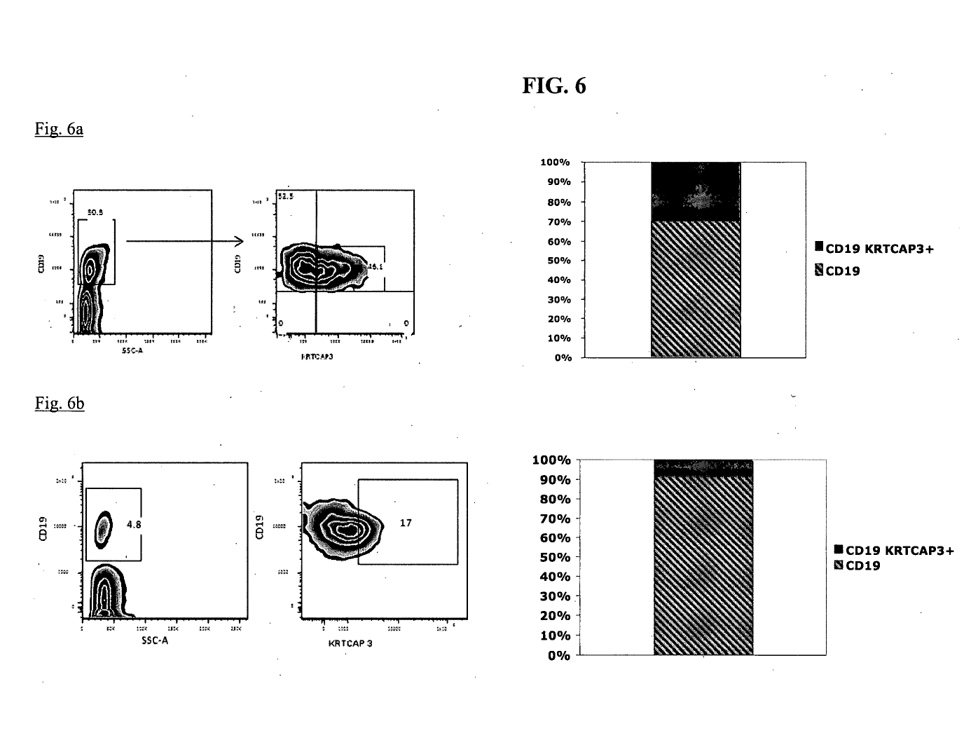

FIG. 6 points out the physiological and medical significance of KRTCAP3 expression in B lymphocytes. The method for obtaining such results is explained in Example 4.

FIG. 6a shows the FACS result of an assay on tonsil samples removed by surgery since they were infected. The FACS result shows that a high percentage of B lymphocytes present (46.1%) express KRTCAP3. The graph on the right shows that on an average of 4 samples of different infected tonsils, 30% of B lymphocytes express KRTCAP3.

FIG. 6b shows the FACS result of an assay on blood samples removed from patients infected with hepatitis C. The FACS result shows that a high percentage of B lymphocytes (17%) present in a sample express KRTCAP3. The graph on the right shows that on an average number of 5 blood samples from different donors infected with hepatitis C, 11% of B lymphocytes express KRTCAP3.

In the context of the present invention, “hematopoietic cells” means all those nucleated cells coming in vivo and/or ex vivo from the dendrogram lineage starting from the hematopoietic stem cell present in bone marrow as far as mature cells such as for instance a mature leukocyte. In the context of the present invention, “activation state” of a cell means its presence in one of the 5 stages of cell cycle known in the art (S, G₀, G₁, G₂ and M) and/or its tendency to start an apoptotic cell cascade and/or the ability of a cell to respond to stimuli and emit stimulating factors or compounds which modify or are part of a physiological response, such as for instance cytokines that are part of an immune response.

In the context of the present invention, “metabolic state” of a cell means the reactions occurring inside a single cell and involved in the production of stimulating proteins/factors/compounds or in the increase of cell number.

In the context of the present invention, the expression of a protein “on the cell surface” means the expression of a protein that gets through the membrane or is anchored to the membrane and shows at least part of its three-dimensional structure on the outer surface of the cell membrane.

In the context of the present invention, “immune response” means any type of physiological response, i.e. a series of biochemical reactions, developed by the host as a result of the contact and/or presence of an antigen with cells belonging to the immune system.

In the context of the present invention, “immune system” means a group of cells and chemical components, among which cytokines, that are present in the hematopoietic system of a mammal. Said cells and chemical components belonging to the immune system can belong to the native or adaptative immune system.

In the context of the present invention, “native immune system” means a part of the immune system making up the first line of defense against infections. It is made up of elements of the organism existing before the infection, acts rapidly but cannot be “instructed” and will therefore respond in the same way when the same infection occurs again. The cells making up the native immune system are cells with phagocytic activity, such as granulocytes, monocytes and macrophages and cells with natural cytotoxic activity (NK).

In the context of the present invention, “adaptative immune system” means a part of the immune system characterized by the ability to discriminate and “recognize” specifically a very large number of different macromolecules (antigens), and by the ability to “remember” an antigen towards which the immune system previously responded. Thanks to these characteristics the adaptative immune system can be instructed and its responses to a re-infection with a pathogen are more rapid and effective. The cells making up adaptative immunity are T lymphocytes and B lymphocytes.

Said components of the immune system and their responses are well known in the art. It is also well known that the various components of the immune system mutually interact to give a complete immune system. In the context of the present invention, the term “cells” includes any maturation stage of said cell, such as e.g. the term “B lymphocytes” includes all possible stages of a B lymphocytes from pro-B cells (CD34⁺CD19⁺CD20⁻Ig⁻) up to a plasma cell for instance (CD38⁺CD27⁺CD19^{+/-}CD20⁻HLA⁻DR⁻).

The object of the present invention are ex vivo hematopoietic cells having/expressing on their surface KRTCAP3. Preferably, said cells are lymphocytes, more preferably B lymphocytes.

The cells according to the invention can derive from any source of hematopoietic cells, preferably from a source of cells belonging to the adaptative immune system and still more preferably in vivo cells. Said source is preferably peripheral blood or a secondary lymphatic tissue.

Preferably, the cells according to the invention derive from a human. Said human is preferably an adult. In an embodiment, the cells according to the invention are included in a composition further comprising excipients and/or stabilizers and/or vehicles. In a preferred embodiment, said composition further comprises a vaccine. In a still more preferred embodiment, said composition further comprises T lymphocytes and/or monocytes. More preferably, PMA and IONO and/or PHA, preferably PHA e IL-2, are further present when T lymphocytes are present in the composition and/or CPG+IL-2 are further present when monocytes are present in the composition.

The cells according to the invention are kept alive ex vivo selecting suitable methods and devices among those known in the art for preserving in vitro hematopoietic cells. In a preferred embodiment, after a separation with FICOLL, the cells are suspended in an isotonic nutrient medium containing salts, vitamins, co-factors and proteins (e.g. media such as RPMI1640 or D-MEM) added with growth factors (e.g. 10% by volume of cultures of Fetal Bovine Serum or Normal Human Serum). When re-suspended in such growth medium, the cells are vital and in good conditions for several hours (up to 24 hours). The advantage of said culture medium is that the cells can also be subjected to various types of stimuli (e.g. treatment with mitogen PHA-L) and their behavior can be monitored for several days, refreshing the culture medium with suitable amounts of fresh medium.

An advantage of cells expressing KRTCAP3 on their surface is that said cells are activated mitogenically and/or stimulated metabolically. Therefore, the cells according to the invention are advantageously used as a drug.

In a preferred embodiment of the use as a drug, the cells according to the invention are used for the preparation of a medicament for treating or preventing diseases or clinical conditions requiring the recovery and/or the increase in the number of hematopoietic cells and/or the increase of effectiveness of hematopoietic cells. In a preferred embodiment, said diseases or clinical conditions are diseases or clinical conditions involving the immune system, still more preferably the adaptative immune system.

In a still more preferred embodiment, the clinical conditions or diseases involve B lymphocytes. An example of said clinical conditions requiring the recovery or an increase in the number of cells belonging to the lymphocyte system are the conditions after lympho-ablative treatment, such as e.g. radiotherapy as a result for diseases such as e.g. leukemia. Another example are the clinical conditions setting up after the administration of a vaccine, increasing the response potential of the immune system to said vaccine. An example of a disease requiring the recovery and/or the increase of effectiveness and/or in the number of hematopoietic cells is anemia or an immunosuppressive disease, such as e.g. DiGeorge syndrome or Wiskott-Aldrich syndrome or AIDS.

Said medicament for recovering and/or increasing the number and/or effectiveness of hematopoietic cells, preferably those belonging to the lymphocyte system, is preferably prepared so as to be administered according to methods known in the art for cell transfusion in a patient. Drug administration in the context of the present invention takes place with methods known in the art, preferably by intravenous injection or direct injection into bone marrow. The drugs prepared according to the invention can be present in a composition as described above.

Another object of the present invention is a method for identifying and/or isolating and/or quantifying the cells according to the invention.

Said method is characterized by at least one step in which the presence of KRTCAP3 on the surface of said ex vivo cells is used to identify and/or isolate and/or quantify the cells according to the invention from or in a sample of cells including hematopoietic cells.

A preferred embodiment of said method includes a step in which the cells are selected either positively or negatively for one or more specific sub-populations of hematopoietic cells. In a preferred embodiment, the cells are selected either positively or negatively for one or more specific sub-populations of cells belonging to the lymphocyte system, such as e.g. B lymphocytes.

In the method for identifying and/or isolating and/or quantifying the cells according to the invention it is preferred to use a ligand for KRTCAP3, more preferably a proteic ligand, such as e.g. an antibody or a protein lectin.

Among said ligands the preferred one is a monoclonal antibody against KRTCAP3. The monoclonal antibody can be, prepared with methods known in the art, such as e.g. recombination methods or methods using Kohler and Milstein's technology. Said method preferably includes the following steps:
i) immunizing an animal having a spleen with protein KRTCAP3 so as to induce an immune response, preferably in combination with an adjuvant;
ii) removing the spleen from the animal and treating it so as to obtain a suspension of intact cells, and isolating from it leukocytes, such as e.g. B lymphocytes;
iii) forming a hybridoma, e.g. by fusion, from a leukocyte cell isolated from the suspension resulting in (ii) with an immortalized cell, such as cells from a lineage myeloma HGRP^{-/-};
iv) enriching the number of cells formed in (iii) with a suitable medium, such as e.g. a cell feeder layer;
v) selecting by a method of negative selection cells that have formed a working hybridoma, such as e.g. growing the cells formed in (iii) on a HAT medium if a myeloma HGRP^{-/-} is used;
vi) isolating cells that produce antibodies against KRTCAP3 by methods known in the art, such as e.g. using KRTCAP3 bound to a marker, e.g. a probe;
vii) isolating and multiplying the selected cells so as to produce monoclonal antibodies against KRTCAP3.

Said ligands can be used in said method for identifying and/or isolating and/or quantifying cells according to the invention from a sample of ex vivo cells in separation protocols known in the art, such as for instance magnetic separation.

Said method for identifying and/or isolating and/or quantifying the cells according to the invention includes the following steps:
preparing a sample of cells comprising hematopoietic cells,
determining the presence of KRTCAP3 on the surface of the cells of said sample with a ligand for KRTCAP3, and
isolating from the sample and/or quantifying and/or identifying the cells on which KRTCAP3 is present.

The method for selecting the cells according to the invention or the specific cell sub-populations can include both positive and negative selection protocols known in the art.

A preferred protocol to be used for identifying and/or isolating said sub-population is a flow cytometry protocol by which the sub-population according to the invention can be isolated by differentiating between cells expressing or not expressing KRTCAP3. Still more preferred is an identification and/or isolation protocol using flow cytometry with fluorochromes (FACS® of Beckton-Dickinson), preferably as a final stage and/or as a stage following an enrichment protocol, such as e.g. a protocol including the use of magnetic beads with specific antibodies bound thereon.

Another object of the present invention is an ex vivo method for detecting the immune or inflammatory state of a patient from a sample of hematopoietic cells of said patient. Preferably, the immune or inflammatory state is due to the adaptative immune state, in particular to the interaction between B and T lymphocytes or between B lymphocytes and monocytes. Said method includes at least one step in which the percentage of B lymphocytes having KRTCAP3 is detected, preferably by means of a ligand for KRTCAP3, which percentage is related to the number of stimulated B lymphocytes, preferably to B lymphocytes stimulated after interactions with T lymphocytes and/or monocytes, preferably after physical interactions with T lymphocytes and/or monocytes.

Another object of the present invention is the use of the ligand according to the invention for preparing a (diagnostic) medicament to be used in a diagnostic or prognostic assay for evaluating the metabolic and/or mitogenic state of hematopoietic cells, preferably of the adaptative immune system. Said diagnostic assay can either be ex vivo or in vivo. In a preferred embodiment, said ligand is bound to a marker, such as for instance a secondary antibody associated to a probe, such as e.g. a fluorescent, phosphorescent or radioactive probe, bound onto the secondary antibody. Said evaluation of the metabolic and/or mitogenic state of the hematopoietic cells can be made as a function of the amount of KRTCAP3 expressed on the surface of one or more cells and as a function of the number and—if in vivo—of the position of cells expressing KRTCAP3 on their surface.

In another embodiment of the invention, said ligand according to the invention can be used for preparing a medicament for modulating an immune response, preferably an immune response involving B lymphocytes.

In a preferred embodiment of said use in the modulation of an immune response, the ligand can be used for preparing a medicament for the prophylaxis or extinction or treatment of auto-immune diseases, such as for instance non-Hodgkin lymphoma or Lupus or inflammatory diseases, such as e.g. dermatitis, asthma, rheumatoid arthritis, vasculitis and psoriasis. Therefore, it is preferred to use antibodies, preferably monoclonal antibodies against KRTCAP3, since these can start an autologous ADCC or CDC cascade to eliminate the lymphocytes they recognize. Preferably, the medicament can contain an adjuvant apt to induce an immune response. Still more preferably, other anti-inflammatory products are further present in the drug.

It is still more preferred if the ligands for KRTCAP3 in said embodiment are bound to harmful substances. The harmful substance is bound to the ligand, such as e.g. by a secondary antibody, and is toxic or at least suitable for eliminating the target of the ligand, i.e. the cell expressing KRTCAP3 on its surface. Said toxic substance can be a toxin or a radioactive atom, such as for instance iodine-131 or an enzyme that may be then involved in a monoclonal therapeutic system known in the art as ADEPT.

In another preferred embodiment of said use in the modulation of an immune response, the ligand for KRTCAP3 can be used for the preparation of a medicament for activating or increasing an immune response involving B and T lymphocytes or for diagnosing the activation or increase of an immune response. The clinical conditions in which an immune response involving B and T lymphocytes should be activated or increased can be in a patient suffering from immunosuppressive diseases or can be when an infection should be prevented with a vaccine or a patient having or risking an infection should be prevented or treated.

Said medicament preferably includes excipients and/or stabilizers and/or vehicles and/or adjuvants. Another object of the invention is the use of stimuli known in the art for activating T lymphocytes or monocytes in the presence of B lymphocytes in order to assay in vitro the ability of T lymphocytes or monocytes to interact with B lymphocytes. Interact preferably means stimulate and/or activate B lymphocytes. Preferably, said stimuli are PMA+IONO and/or PHA, preferably PHA+IL2 when T lymphocytes are present, and CPG+IL2 when monocytes are present.

Preferably, the stimuli with the combination of cells are left for at least 24 hours, preferably at least 36 hours, preferably at least 42 hours, more preferably at least 48 hours.

In a preferred embodiment, a medium, suitably selected by a skilled technician, containing B lymphocytes and T lymphocytes and/or monocytes is used and the stimuli known in the art are added thereto for activating T lymphocytes or monocytes. After 24 hours only stimulated B lymphocytes are obtained, which can be identified by KRTCAP3 expression as described above. The level of interaction between T lymphocytes and/or monocytes and B lymphocytes can be determined as a function of KRTCAP3 expression on B lymphocytes after 24 hours.

Said assay can advantageously determine a mitogenic and/or metabolic activation and/or stimulation from monocytes and/or T lymphocytes on B lymphocytes. The assay can advantageously give a result in less than 24 hours, preferably in less than 36 hours, preferably in less than 42 hours, more preferably in less than 45 hours and still more preferably in less than 48 hours. The assay does not advantageously require the presence of specific antigens for activating monocytes and/or T lymphocytes. Thanks to said advantages the assay is less complex, less expensive and faster than the assays known in the art, such as e.g. “Helper T-Cell assay” as the one described in Galli G., Nuti S., Tavarini S., Galli-Stampino L., De Lalla C., Casorati G., Dellabona P., Abrignani S., CD1d-restricted help to B cells by human invariant natural killer T lymphocytes. J Exp Med. 2003 Apr. 21; 197(8): 1051-7. Epub 2003 Apr. 14, wherein it takes at least 5 days (see paragraph 9, first column, page 1052 and FIG. 1).

Another object of the invention is the use of T lymphocytes and/or monocytes in presence of B lymphocytes for assaying in vitro the ability of compounds to activate or stimulate T lymphocytes and/or monocytes in an adaptative immune response. The ability of the compound to activate or stimulate T lymphocytes and/or monocytes is determined by the presence of KRTCAP3 on the surface of B lymphocytes. The compound is preferably a chemical compound which a skilled technician can reasonably deem as working as an antigen, such as e.g. a proteic sequence.

The skilled technician can use protocols, methods and devices as described above for implementing said assay. The assay can advantageously determine whether the compound activates or stimulates monocytes or T lymphocytes as a function of which of both subpopulations of cells is present in the assay together with B lymphocytes. The assay can be advantageously implemented in less than 24 hours, preferably in less than 36 hours, preferably in less than 42 hours, more preferably in less than 45 hours and still more preferably in less than 48 hours.

### EXAMPLE 1

### Relation Between the Presence of KRTCAP3 and the Metabolic and Mitogenic State of Hematopoietic Cells According to Invention and Distribution in Some Sub-Populations Thereof

A. Isolation of Mononucleated Cells from Peripheral Blood
1. A 10 ml sample of blood from a healthy donor was diluted 1:3 in a phosphate buffered saline solution (PBS).
2. 15 ml of Ficoll-Hypaque (density 1.077 g/l) were introduced into a 50 ml Falcon tube and then 30 ml of peripheral blood from a healthy donor was layered thereon. Blood was dropped very slowly so as not to perturb the interface. The operation was repeated until the whole sample was over.
3. The Falcon tube was then centrifuged at 1600 rpm for 30 min. at room temperatures without braking. Mononucleated cells (PBMCs) lay on the interface between Ficoll-Hypaque and plasma. Said PBMC ring was collected and transferred into a 50 ml Falcon tube.
4. PBMCs were washed twice with 50 ml PBS containing 5% normal human serum (NHS) centrifuging for 10 min. at 1200 rpm.
5. The pellet was then washed with 50 ml PBS 5% NHS centrifuging for 10 min. at 800 rpm.
6. The PBMCs resulting in a pellet at the end of step 5 were re-suspended in 10-30 ml PBS 5% NHS at room temperature.
B. Isolation of Cells According to the Invention from Non-Stimulated PBMC
1. The cells were counted with a Bunker chamber and 0.3 to 0.5×10⁶ PBMCs per sample were colored.
2. The samples were incubated for 20 min. at room temperature with PBS 50% NHS.
3. The samples were centrifuged for 3 min. at 1500 rpm and, without washing, were incubated for 10 min. in an ice bath with antiserum KRTCAP3 diluted 1:50 in 100 microliters PBS 5% NHS.

The antiserum KRTCAP3 was prepared with methods known in the art, immunizing mice with the primary structure of KRTCAP3.

Samples for negative control were incubated for 10 min. in ice with antiserum of a non-immunized mouse for setting the negativity of the final color of the image resulting from FACS.

4. The cells of the centrifuged samples were washed twice with PBS 5% NHS, removing the supernatant after centrifugation for 3 min. at 1500 rpm and resuspending with PBS 5% NHS.
5. Said re-suspended cells were then incubated again for 10 min. in an ice bath with Goat-anti-mouse IgG-PE (Southern Biotech®), a known “secondary” antibody with fluorochrome phycoerithrin (PE) bound thereon, diluted 1:100 in 100 microliters PBS 5% NHS.
6. The cells were then washed twice with PBS 5% NHS, centrifuging for 3 min. at 1500 rpm and re-suspending with PBS 5% NHS.
7. The re-suspended pellet was added with 12 micrograms per sample of mIgG (mouse immunoglobulines) and incubated for at least 60 min. in ice.
8. The cells were incubated for 10 min. in an ice bath with m-apha-hCD19Cychrome (BD Biosciences®), a known monoclonal antibody with the fluorochrome PE-Cy5 bound thereon, with mouse-anti-hCD3FITC (BD Biosciences), a known monoclonal antibody with fluorochrome fluorescein (FITC) bound thereon, with mouse-anti-hCD56APC (BD Biosciences®), a known monoclonal antibody with fluorochrome allophycocianin bound thereon, and with mouse anti human CD69PE-Cy7 (BD Biosciences®), a known monoclonal antibody with fluorochrome PE-Cy7 bound thereon.
9. Eventually, the colored cells were washed (centrifuging at 1500 rpm for 3 min.) with PBS 10% NHS and re-suspended in 500 microliters for acquisition with FACSCalibur®.
10. The Beckton-Dickinson-FACS® machine was operated according to protocols known in the art and quoted in Current Protocols in Immunology (2001), John Wiley and Sons Inc., Units 5.4.1-5.4.22 for giving the obtained results, as shown in FIG. 1, FIG. 1a) i).

Non-stimulated PBLs are identified thanks to Forward-Scatter (FSC) and Side-Scatter (SSC) features and thanks to the expression of markers CD3, CD19 and CD56.

It can be observed that the population of non-stimulated PBL cells does not substantially express KRTCAP3 (the presence of the small peak is highly variable and has no statistical significance).

C. Isolation of Cells According to the Invention from Stimulated PBMC
1. A part of the cells obtained after separation with Ficoll as described in Example 1A are sown in U-bottom, 96-well cell culture plates at a concentration of 0.5×10⁶/ml in 200 ml of culture medium containing 1μg/ml of Phytohemagglutinin-L (PHA-L, Roche).
2. The cells are incubated in presence of PHA mitogen for 48 hours.
3. After incubation with the mitogen the cells are taken up and colored so as to assess the presence of KRTCAP3 as described in 15-17 of Example 1B. The results obtained are shown in FIG. 1, FIG. 1a)ii) and FIG. 1b.

The results point out that after incubation with PHA at 48 hours, 5.14% of PBL cells express KRTCAP3. When a selection for specific lymphocytes is made, it can be observed that the relative numbers of cells having KRTCAP3 on the surface of T lymphocytes and NK cells are much smaller than the number of the same cells not expressing KRTCAP3, so that these can be due to a background noise caused by the method for the preparation of antibodies for KRTCAP3 used in FACS test and have therefore no statistical significance. Conversely, it should be noted that the number of B lymphocytes (CD 19⁺) expressing KRTCAP3 is very large; 35% (2.51/(4.73+2.51)×100) of B lymphocytes present. In order to verify the indication on KRTCAP3 expression given by FACS test described above, a control test was made with RT-PCR so as to monitor the expression of KRTCAP3 gene first in non-stimulated peripheral blood mononucleated cells compared with PHA-stimulated cells. To this purpose RNA was extracted with Qiagen kit (cat# 74104) from cells purified by means of Ficoll, according to the supplier's protocol, and cDNA was prepared from 100 ng of RNA by means of RetroScript enzyme (Ambion, cat# 1710), according to the supplier's protocol.

2 μl of cDNA were used for RT-PCR analysis by means of specific primers for KRTCAP3. RT-PCR for beta-actin gene was executed as positive control, since beta-actin is a protein which—as is known—is expressed by all cells. The following primers were used:

| | |
|---|---|
| KRTCAP3 fw: | SEQ ID NO. 1 |
| KRTCAP3 rev: | SEQ ID NO. 2 |

Gene of beta-actin fw: SEQ ID NO. 3

Gene of beta-actin rev: SEQ ID NO. 4

The full sequences are listed in the attachment according to WIPO ST.25 international standard and developed with Patent-In 3.3 software.

The following conditions for RT-PCR with specific primers for KRTCAP3 were used:

cDNA: 2 microliters
SEQ ID NO. 1 (10 microM): 1 microliter
SEQ ID NO. 2 (10 microM): 1 microliter
2X Taq PCR Master Mix (Qiagen, cat #201443): 25 micro-liters
Sterile water: up to a final volume of 50 microliters.

Conditions of PCR thermal cycles:

### 94° C., 3 min.

30 cycles at 94° C. for 30 sec.; 55° C. for 30 sec. and

### 72° C. for 30 sec.

### 72° C., 10 min.

### ∞, 4° C.

A similar PCR reaction was executed with beta-actin primers.

The results are shown in FIG. 1c where there is an increase of KRTCAP3 expression in PBMC after stimulation with PHA.

D. Isolation of B Lymphocytes According to the Invention from Stimulated PBMC

The procedure of step C was repeated after incubating the mixture of PBMC cells with 1 μg/ml PHA (PHA-L, Roche) in the presence of 100 U/ml IL-2 (recombinant human IL-2, Chiron) for 48 hours. The results obtained are shown in FIG. 2.

The results confirm what had already observed after activation with PHA. A percentage of PBL cells, 4.13%, express KRTCAP3. Substantially the whole of this percentage is made up of B lymphocytes (95.5%). The remaining percentage can be due to, the background noise caused by FACS method.

### EXAMPLE 2

### Relation Between the Presence of KRTCAP3 and the Metabolic and Mitogenic State of B Lymphocytes

### A. Analysis of KRTCAP3 Expression on Activated B Lymphocytes

1. Peripheral blood mononucleated cells (PBMCs), isolated by means of Ficoll as described in Example 1A, are plated in U-bottom 96-well plates (5×10⁵ cells per well) and stimulated under the following conditions:
1 μg/ml PHA (PHA-L, Roche) in the presence of 100 U/ml IL-2 (recombinant human IL-2, Chiron)
25 ng/ml PMA (SIGMA) in the presence of 300 ng/ml Ionomycin (SIGMA)
5 μg/ml GPG (PRIMM) in the presence of 1000 U/ml IL-2 (recombinant human IL-2, Chiron)
5 μg/ml SAC (Pansorbin cells, Chiron)
5 μg/ml anti human IgM (BD Biosciences®)
no stimulus (negative control)
   2. The cells are incubated in the presence of the stimuli for 48 hours.
   3. The cells are taken, colored and analyzed as described in 2-10 of Example 1B.

The results obtained are shown in FIG. 3, FIG. 3a. It can be observed from the resulting numbers that the percentage of B lymphocytes expressing KRTCAP3 highly increases as a function of specific metabolic and/or mitogenic cell stimuli that are not directed specifically towards B lymphocytes. As a matter of fact, anti IgM and SAC stimuli, which are specific for B lymphocytes, do not induce a significant increase of KRTCAP3 expression. Conversely, a high, statistically significant increase of KRTCAP3 expression on activated. B lymphocytes can be noted when using stimuli that are more specifically directed towards T lymphocytes (such as PHA IL-2) or monocytes (such as CPG IL-2).

B. Analysis of KRTCAP3 Expression on Stimulated B Lymphocytes after Isolation from PBMC or in the Presence of T Lymphocytes Only or Monocytes Only

Peripheral blood mononucleated cells (PBMCs), isolated by means of Ficoll as described in Example 1, are subjected to three purification processes:
i) Peripheral blood B lymphocytes are purified from PBMC by using “B cell isolation” kit (Miltenyi Biotech), according to the supplier's protocol.
ii) In order to obtain a culture of T and B lymphocytes only, monocytes are removed from PBMC by magnetic separation with “CD14 microbeads” kit (Miltenyi Biotech).
iii) In order to obtain a culture of B lymphocytes and monocytes only, T lymphocytes are removed from PBMC by magnetic separation with “CD3 microbeads” kit (Miltenyi Biotech).

The populations thus obtained are plated in U-bottom 96-well plates (5×10⁵ cells per well) and stimulated under the following conditions:
1 μg/ml PHA (PHA-L, Roche) in the presence of 100 U/ml IL-2 (recombinant human IL-2, Chiron)
25 ng/ml PMA (SIGMA) in the presence of 300 ng/ml Ionomycin (SIGMA)
5 μg/ml GPG (PRIMM) in the presence of 1000 U/ml IL-2 (recombinant human IL-2, Chiron)

The cells are incubated in the presence of the above stimuli for 48 hours and then taken, colored and analyzed as described in 2-10 of Example 1B.

The results obtained are shown in FIG. 3, FIG. 3b. It can be noted that B lymphocytes isolated from other sub-populations of PBMC, though activating as a response to the stimuli used as shown by the increase of the expression of CD69 marker, are not able to significantly increase the expression of the protein KRTCAP3 on their surface as a response to such stimuli. It can also be noted that B lymphocytes grown in the presence of T lymphocytes only increase KRTCAP3 expression as a response to PMA IONO but not to CPG IL2, whereas B lymphocytes grown in the presence of monocytes only increase KRTCAP3 expression as a response to CPG IL2 but not to PMA IONO. These results indicate that KRTCAP3 expression on activated B lymphocytes is the result of an interaction between B and T lymphocytes if the stimulus used is PHA IL-2 or PMA IONO, or between B lymphocytes and monocytes if the stimulus used is CPG IL-2.

C. Demonstration that a Physical Connection of T Lymphocytes and/or Monocytes is Necessary to Activate B Lymphocytes

Following the method described in Example 2A and 2B, B lymphocytes were activated either in the presence of or without other PBMC cells with PHA+IL-2. The results were analyzed with FACS to determine their mitogenic activation.

The results of the tests are shown in FIG. 5.

FIG. 5a shows a series of tests executed on B lymphocytes with other PBMC cells present.

Without stimuli after 48 hours there is no KRTCAP3 expression, but after stimulation with PHA+IL-2 there is a large presence of KRTCAP3. As an alternative, the cells are stimulated with a supernatant. The supernatant is obtained by stimulating PBMC cells with PHA+IL-2 for 48 hours. After such a period the cells are centrifuged and the culture medium without cells is taken up. Said supernatant contains all soluble factors which the cells produce as a response to a stimulation with PHA+IL-2, but not the cells themselves, and is then used to stimulate isolated B lymphocytes deriving, from a different blood sample.

The results shown in FIG. 5 show that the supernatant does not induce KRTCAP3 expression on B lymphocytes. Therefore, FIG. 5a shows that the physical presence of other PBMC cells is necessary to activate B lymphocytes with PHA+IL-2, and not only soluble factors such as e.g. cytokines.

FIG. 5b shows the negative control, in which isolated B lymphocytes are stimulated without PBMC and thus without T lymphocytes. Under such conditions no stimulus induces KRTCAP3 expression.

### EXAMPLE 3

### Demonstration of a Direct Relation Between KRTCAP3 Presence and the Mitogenic State of B Lymphocytes

In this test the methods described in Example 2A were used to demonstrate that after activation with PHA+IL-2, but not with SAC, B lymphocytes express KRTCAP3. In an independent manner the mitogenic activity of said B lymphocytes was demonstrated by means of an assay known in the art, which uses coloring agent CFSE-A (5,6-carboxyfluorescein diacetate succinimidyl ester).
1. Peripheral blood mononucleated cells (PBMC), isolated by means of Ficoll as described in Example 1A.
2. PBMCs thus obtained are brought to a concentration of 20×10⁶ cells/ml and incubated for 10 minutes at room temperature with a solution 1 mM of CFSE-A (Molecular Probes)
3. The cells are plated in U-bottom 96-well plates (5×10⁵ cells per well) and stimulated under the following conditions:
   1 μg/ml PHA (PHA-L, Roche) in the presence of 100 U/ml IL-2 (recombinant human IL-2, Chiron)
   5 μg/ml SAC (Pansorbin cells, Chiron)
4. The cells are analyzed after 24 hours from the beginning of stimulation to evaluate the fluorescence emission intensity of coloring agent CFSE-A before the cells start any mitogenic activity.
5. The cells are then analyzed again after 5 days and it is now possible to make a quantitative analysis of any mitogenic activity, which can be inferred from the presence of CFSE-A emission peaks at lower fluorescence intensity. As a matter of fact, since the coloring agent CFSE-A is vital, as the cells divide the amount of coloring agent present in the cells dilutes because it is divided among the daughter cells in every division cycle. As a result, whenever a cell divides its fluorescence emission of CFSE-A is reduced. By means of FlowJo software (Treestar) it is possible to make a quantitative analysis of fluorescence reduction of CFSE-A on a given cell population after stimulation.

The results are shown in FIG. 4.

FIG. 4a clearly shows that FACS results demonstrate a KRTCAP3 expression in B lymphocytes only after stimulation with PHA and IL-2.

Conversely, FIG. 4b shows the mitogenic activity as detected by coloring agent CFSE-A. The middle column shows the mitogenic activity of B lymphocytes not expressing KRTCAP3, whereas the right-hand column shows B lymphocytes expressing KRTCAP3. It can be noted that B lymphocytes stimulated with PHA-IL-2 show a mitogenic activity and, as can be seen from stimulation after 5 days, B lymphocytes expressing KRTCAP3 show a higher mitogenic activity than B lymphocytes not expressing KRTCAP3.

The mitogenic activity of B lymphocytes determined with CFSE-A, 24 hours and 5 days after stimulation with SAC, is shown as positive control of the method of analysis with CFSE-A.

A quantitative analysis of CFSE-A peaks, executed with Flow Jo software, points out the following proliferation indexes for B lymphocytes stimulated with PHA+IL-2:

| B lymphocytes not expressing | B lymphocytes expressing |
|---|---|
| KRTCAP3 | KRTCAP3 |
| 37.6% have divided | 58.6% have divided |
| Show a division index | Show a division index |
| of 0.55 | of 1.87 |
| Show a proliferation | Show a proliferation |
| index of 1.47 | index of 3.19 |

Therefore, it can be clearly observed that cells expressing KRTCAP3 are mitogenically more active than those not expressing KRTCAP3.

### EXAMPLE 4

### Demonstration of KRTCAP3 Presence and the Immune State of in vivo B Lymphocytes

As shown in Example 1 and in FIG. 1, if hematopoietic cells and in particular B lymphocytes are not stimulated, they do not substantially express KRTCAP3. Said example demonstrates that, when necessary, i.e. under a physiological need of the immune system, B lymphocytes, being part of the adaptative immune system, activate and express KRTCAP3 also in vivo.

1 sample was taken from 4 different infected tonsils removed by surgery. Tonsils are a secondary lymphoid tissue but have been specifically selected because they are infected and thus indicative of the state of B lymphocytes active in an immune system during an acute inflammatory stage.

The lymphoid tissue is disgregated down by making it get through a suitable strainer. Then the procedure described in Example 1A is followed.

Then a FACS study was executed as described in Example 1B. The results are shown in FIG. 6b and the average value of the four samples clearly shows that 30% of B lymphocytes express KRTCAP3, a significant number with respect to their absence under normal conditions. 5 samples of peripheral blood have also been taken from 5 different donors infected with hepatitis C virus, since their blood is indicative of the state of B lymphocytes active in an immune system fighting against an infection.

The protocol of Example 1A and 1B was executed obtaining FACS results as shown in FIG. 6B. The average value of the 5 different donors clearly shows that a significant percentage thereof, 11%, express KRTCAP3.

1.-18. (canceled)

19. Ex vivo hematopoietic cells characterized by the expression of KRTCAP3 on the surface of said cells.

20. The cells according to claim 1, wherein the cells are B lymphocytes.

21. A composition comprising the cells according to claims 1.

22. The composition according to claim 3, wherein T lymphocytes and/or monocytes are further present.

23. The composition according to claim 4, wherein PMA and IONO and/or PHA or PHA+IL-2 are further present when T lymphocytes are present in said composition, and/or wherein CPG+IL-2 are further present when monocytes are present in said composition.

24. A method for identifying and/or isolating and/or quantifying the cells according to claim 1, including the following steps:
a. preparing a sample of cells comprising hematopoietic cells;
b. determining the presence of KRTCAP3 on the surface of the cells of said sample with a ligand for KRTCAP3; and
c. isolating from the sample and/or quantifying and/or identifying the cells on which KRTCAP3 is present.

25. The method according to claim 6, wherein there is a further a step of selection for lymphocyte cells.

26. Method for treating or preventing diseases or clinical conditions requiring the recovery and/or increase of the number and/or the increase of effectiveness of hematopoietic cells, comprising administering an effective amount of cells according to claim 1 to a patient in need thereof.

27. Method according to claim 8, wherein said cells are cells belonging to the lymphocyte system.

28. Method according to claim 8, wherein said diseases or clinical conditions are anemia or immunodepressive diseases.

29. An ex vivo method for detecting the immune or inflammatory state of a patient from a sample of hematopoietic cells of said patient, including at least one step in which the percentage of B lymphocytes having KRTCAP3 is detected.

30. Method for evaluating the metabolic and/or mitogenic state of the hematopoietic system of a patient, comprising contacting, ex vivo or in vivo, a ligand for KRTCAP3 with said hematopoietic system and evaluating the amount of KRTCAP3 expressed on the surface of cells.

31. Method according to claim 12, wherein said hematopoietic system is the lymphocyte system.

32. Method according to claim 12, wherein the ligand is bound to a marker.

33. Method for modulating the immune response in a patient comprising administering a ligand for KRTCAP3 to a patient in need thereof.

34. Method according to claim 15, wherein the ligand is bound to a harmful substance.

35. Method according to claim 15, wherein the modulation of the immune response is used in the prophylaxis or treatment of auto-immune diseases or inflammatory diseases.

36. Method for activating T lymphocytes or monocytes in the presence of B lymphocytes for assaying in vitro the ability of T lymphocytes or monocytes to interact with B lymphocytes, comprising contacting PMA+IONO and/or PHA, and/or PHA+IL2 with said T lymphocytes or monocytes.

37. Method for assaying in vitro the ability of compounds to activate or stimulate T lymphocytes and/or monocytes in an adaptative immune response, comprising contacting T lymphocytes or monocytes in the presence of B lymphocytes with said compounds.
